(19) United States
(12) Patent Application Publication
(10) Pub. No.: US 2004/0049225 A1
(43) Pub. Date: Mar. 11, 2004

(54) Aspiration catheter

(76) Inventor: Andy E. Denison, Temecula, CA (US)

Correspondence Address: FULWIDER PATTON LEE & UTECHT, LLP, HOWARD HUGHES CENTER, 6060 CENTER DRIVE, TENTH FLOOR, LOS ANGELES, CA, 90045, US

(21) Appl. No.: 10/241,160

(22) Filed: Sep. 11, 2002

(51) Int. Cl. A61M 29/00

(52) U.S. Cl. 606/200; 606/194

(57) ABSTRACT

A catheter comprising an elongated shaft having a first lumen extending from a proximal shaft section to the distal end of the shaft with a distal port at the shaft distal end, and a second lumen extending in at least a distal shaft section from a proximal port to a distal port located proximal to the distal port of the first lumen. In a one embodiment, the catheter is an aspiration catheter with a vacuum source in fluid communication with the first lumen, providing improved removal of embolic debris from within a body lumen. The first lumen can alternatively be connected to a source of fluid, so that the catheter is a fluid delivery catheter such as perfusion or drug delivery catheters.

### BACKGROUND OF THE INVENTION

This invention generally relates to catheters, and particularly intravascular aspiration catheters.

Release of embolic debris during treatment of diseased blood vessels is a significant and potentially deadly problem. For example, pieces of a lesion in an occluded blood vessel may become dislodged during treatment of the occlusion during a balloon angioplasty procedure. In balloon angioplasty, a dilatation catheter having an inflatable balloon on a distal shaft section is advanced into the patient's vessel until the balloon is properly positioned across the lesion, and the dilatation balloon is inflated one or more times to a predetermined size so that the lesion is compressed against the arterial wall and the wall expanded to open up the vascular passageway. Dislodged pieces of the lesion can move downstream and completely block another portion of the blood vessel, thus causing myocardial infarction when used in the coronary anatomy or a stroke when used in the neural or carotid anatomy. Similarly, during delivery and deployment of an intravascular prosthesis such as a stent used to strengthen the dilated vessel, the stent struts may sheer off pieces of the lesion. In an atherectomy procedure in which the lesion is cut away from the blood vessel wall by the mechanical cutting apparatus of the atherectomy catheter, failure to capture and remove all the biological debris from the blood vessel can similarly result in an embolic event. Additionally, during treatment of diseased vessels by laser ablation in which the lesion is vaporized, one difficulty has been ensuring complete vaporization of all the biological material dislodged during the process.

Embolic protection devices, which have been developed to address the problem of capturing and removing embolic debris, include a filter or occlusion balloon placed downstream from the treatment site to trap embolic debris before it reaches the smaller blood vessels downstream. However, there have been problems associated with filtering systems. For example, the filter can become clogged with debris, so that blood circulation past the clogged filter will be insufficient for the downstream vessels and organs. If a filter should become clogged when in use in the carotid arteries, blood flow could be diminished to the vessels leading to the brain, and the physician administering the procedure may be unaware that the filtering device is clogged and that there is little or no blood flowing to the brain. Similarly, the debris trapped by the occlusion balloon must be completely removed from the blood stream to avoid the potential for injury to the patient. Aspiration or vacuum catheters have been suggested for removing embolic debris by suction of the debris from the bloodstream. However, there have been complications with such systems. The aspiration catheter may not always remove all of the embolic material from the bloodstream, and overly powerful suction could cause problems to the patient's vasculature.

Accordingly, it would be a significant advance to provide a catheter providing improved embolic protection during treatment of a stenosed blood vessel. This invention satisfies these and other needs.

### SUMMARY OF THE INVENTION

The invention is directed to a catheter which has an elongated shaft having a first lumen extending from a proximal shaft section to the distal end of the shaft with a distal port at the shaft distal end, and having a second lumen extending in at least a distal shaft section to a distal port located proximal to the distal port of the first lumen.

In a presently preferred embodiment, the catheter is an aspiration catheter with a vacuum source in fluid communication with the first lumen, providing improved removal of embolic debris from within a patient's body lumen. In accordance with the invention, the distal most end of the catheter defines the distal port of the aspiration lumen (i.e., first lumen), and facilitates positioning the aspiration port as close as possible to the embolic debris to be removed. Although discussed primarily in terms of an aspiration catheter, it should be understood that the first lumen can alternatively be connected to a source of fluid, so that the catheter is configured for fluid delivery, as for example as a perfusion or drug delivery catheter, or the delivery of contrast media used to visualize the anatomy under x-ray.

The second lumen is configured to slidably receive a device such as a guidewire or an embolic protection device. In use, the catheter is typically advanced over the previously introduced device within a patient's body lumen until the distal end of the catheter is positioned at the desired location within the body lumen. In a presently preferred embodiment, the device over which the catheter is advanced is an embolic protection device such as an occlusion balloon catheter or a filter catheter having a trap or filter on a distal section thereof. However, the catheter of the invention may be used with a variety of conventional embolic protection devices, see for example U.S. Pat. Nos. 6,398,756 and 6,383,206, incorporated by reference herein, for details regarding balloon occlusion and filter type embolic protection devices.

The distal end of the catheter of the invention, which defines the distal port of the first lumen, is configured to facilitate aspiration of embolic debris from around and within the embolic protection device. Thus, embolic debris which is otherwise difficult to access can be removed from within the body lumen by positioning the catheter distal end, which has a specially configured shape and which defines the distal port of the aspiration lumen, directly at the location of the debris. For example, in one embodiment, the distal end of the catheter has a wedge or truncated shape configured to fit in the space between the edge of an expanded occlusion balloon and the blood vessel wall. The distal end of the catheter can have a variety of suitable shapes, and in one embodiment the shape of the distal end of the catheter is selected from the group consisting of truncated, tapered, and squared, depending on the embolic protection device used with the catheter of the invention.

In a presently preferred embodiment, the catheter of the invention is a rapid exchange type catheter, so that the second lumen is a relatively short lumen extending in the distal shaft section from a proximal port located distal to the proximal end of the shaft to the distal port located proximal to the distal port of the first lumen. However, in an alternative embodiment, the catheter is an over-the-wire type catheter in which the second lumen proximal port is located at the shaft proximal end. In one embodiment, a support mandrel extends along at least the proximal shaft section, for improving the pushability of the catheter.

The catheter of the invention provides for improved removal of embolic debris trapped by an embolic protection device. Due to the configuration of the ports of the first and second lumens, the distal aspiration port can be positioned in a desired location in a patient's body lumen for removal of embolic debris from around or within an embolic protection device, to thereby prevent or inhibit debris in the bloodstream from causing a blockage in vessels at downstream locations or a blockage of blood flow through filtering devices. Moreover, the catheter of the invention provides a system and method which is easy for a physician to use. These and other advantages of the invention will become more apparent from the following detailed description of the invention and accompanying exemplary drawings.

### BRIEF DESCRIPTION OF THE DRAWINGS

FIG. 1 is an elevational view, partially in section, of a catheter which embodies features of the invention, with the catheter over a balloon occlusion catheter in a patient's body lumen.

FIG. 2 is a transverse cross sectional view of the catheter shown in FIG. 1, taken along line 2-2.

FIG. 3 is a transverse cross sectional view of the catheter shown in FIG. 1, taken along line 3-3.

FIG. 4 is a transverse cross sectional view of the catheter shown in FIG. 1, taken along line 4-4.

FIG. 5 illustrates the distal section of an alternative embodiment of the catheter of FIG. 1, with a tapered distal end, and with the catheter over a filter embolic protection device.

FIG. 6 illustrates the distal section of an alternative embodiment of the catheter of FIG. 1, with a squared distal end.

### DETAILED DESCRIPTION OF THE INVENTION

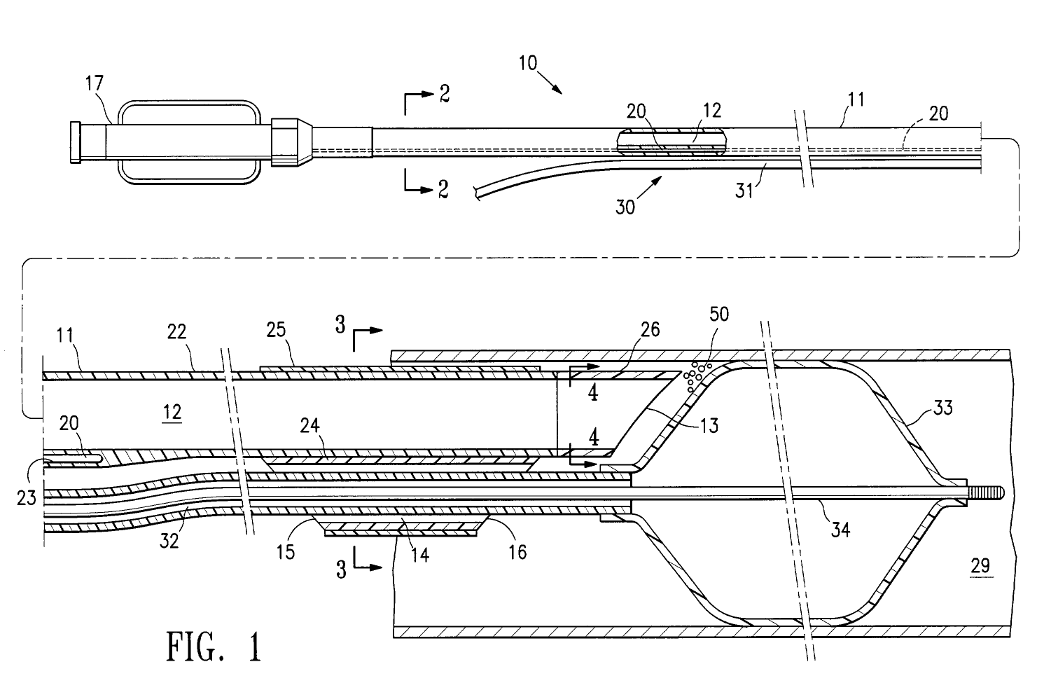

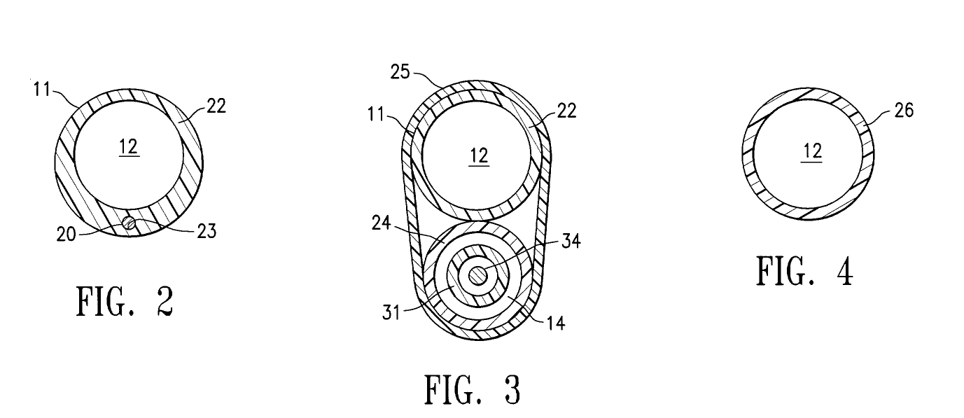

FIG. 1 illustrates a rapid exchange type catheter 10 embodying features of the invention. Catheter 10 generally comprises an elongated catheter shaft 11 having a proximal end, a distal end, a first lumen 12 extending from the proximal to the distal end of the shaft with a distal port 13 at the shaft distal end. A second lumen 14 extends in a distal shaft section from a proximal port 15 located distal to the proximal end of the shaft to a distal port 16 located proximal to the distal port 13 of the first lumen 12. In one embodiment, the distal port 16 of the second lumen 14 is spaced about 1 to about 30 mm, preferably about 10 to about 20 mm proximally from the distal port 13 of the first lumen 12. An adapter 17 on the shaft proximal end is configured to provide access to the first lumen 12. In the embodiment of FIG. 1, the catheter 10 is an aspiration catheter, and is positioned in a patient's body lumen 29 for aspiration of material from within the body lumen. The adapter 17 is configured for connecting a vacuum source (not shown) to the aspiration catheter 10, in fluid communication with the first lumen 12, for aspiration through the first lumen 12. The catheter 10 may alternatively be used as a fluid delivery catheter, so that the adapter 17 may be configured for connecting a fluid source (not shown) to the first lumen for delivery of fluid through the first lumen to the body lumen 29. Although not illustrated, the adapter 17 may have multiple arms for connecting to vacuum and fluid sources. FIGS. 2-4 illustrate transverse cross sectional views of the catheter 10, taken along lines 2-2, 3-3, and 4-4, respectively. In the embodiment of FIG. 1, the first lumen 12 has an inner diameter larger than the inner diameter of the second lumen 14. The inner diameter of the first lumen 12 is typically about 0.25 to about 2 mm, preferably about 0.76 to about 1.14 mm, and the inner diameter of the second lumen 14 is typically about 0.25 to about 1.4 mm, preferably about 0.44 to about 1.1 mm. In an alternative embodiment (not shown), the second lumen 14 is larger than the first lumen 12.

In the embodiment of FIG. 1, a support mandrel 20 extends in a proximal shaft section from the proximal end of the shaft to a location spaced proximal to the proximal port 15 of the second lumen 14. In alternative embodiment, the support mandrel distal end may be located distal to the proximal port 15 of the second lumen 14, including for example being located between the proximal port 15 and the distal port 16 of the second lumen 14. The support mandrel is preferably a solid rod or wire, and is preferably formed of a high strength, flexible material, including metallic materials such as stainless steel, NiTi alloy, MP35N, and cobalt chrome (L605). The support mandrel 20 is in a wall of the shaft 11, and preferably in a mandrel lumen in the wall, radially adjacent (i.e., alongside) the first lumen 12 in the proximal shaft section. The support mandrel 20 is configured to increase the pushability of the catheter shaft 11, and typically has a length which is about 40 to about 100% of the length of the catheter shaft 11.

In the embodiment of FIG. 1, the catheter 10 comprises a first polymeric tubular member 22 defining the first lumen 12, and a second polymeric tubular member 24 defining the second lumen 14 and secured to the distal section of the first polymeric tubular member 22, as for example by heat shrink tubing 25 therearound as best illustrated in FIG. 2. The first polymeric tubular member 22 is formed by extruding the tube with the first lumen 12 extending from the proximal to the distal end thereof, and with a blind lumen in a proximal section thereof configured to receive the mandrel 20. Although illustrated in FIG. 2 as tightly fitting within the mandrel lumen, the mandrel lumen is typically sufficiently large to facilitate sliding the mandrel into the lumen. The mandrel 20 may be secured in place in the mandrel lumen as for example by adhesive or heating the polymeric tubular member 22 therearound, or attached at the mandrel proximal end only, or merely contained in the mandrel lumen and not fixed to the shaft. However, a variety of suitable methods can be used to form the catheter 10, as are conventionally known, including for example by extruding a tubular member with the lumens 12, 14 therein. The first and second polymeric tubular members 22, 24 may be formed of the same or different polymeric materials. Although the first polymeric tubular member 22 defining the first lumen 12 is illustrated as a single length of tubing, it should be understood that multiple longitudinal sections of tubing joined together along the length of the catheter 10 can be used, as for example to provide variable or increasing flexibility along the length of the catheter. Additionally, although illustrated in FIG. 3 with a space between the outer tubular member 25 and the tubular members 22, 24, the space may be filled in with polymeric material as for example by polymeric material from the tubular members 22, 24, 25 flowing into the space during heat bonding thereof. Similarly, although illustrated with circular transverse cross sections a variety of shaft configurations may be used as are conventionally known including semi-circular, oblong, crescent shaped and the like.

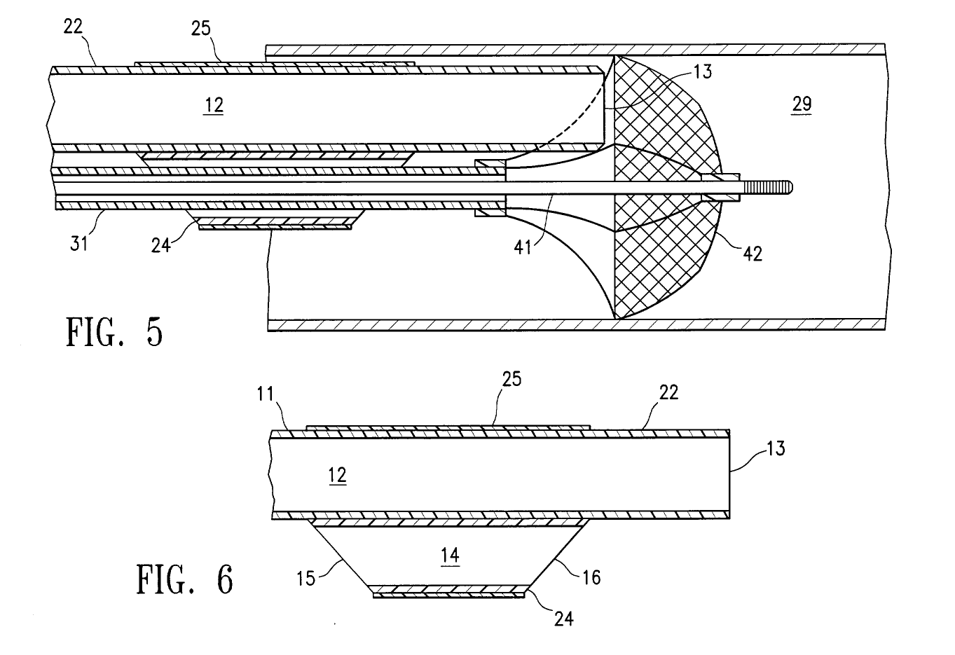

The second lumen 14 of catheter 10 is configured to slidably receive a device therein, over which the catheter is advanced within the body lumen 2, so that the second lumen 14 is open to outside the catheter 10 (i.e., the second lumen distal port 16 is open to allow the catheter 10 to be advanced over a device). The catheter 10 is illustrated in FIG. 1 with an embolic protection device 30 slidably disposed in the second lumen 14. A variety of suitable conventional devices 30 may be used with the catheter 10 of the invention. In the embodiment of FIG. 1, the embolic protection device 30 is a balloon occlusion catheter comprising an elongated shaft 31 defining an inflation lumen 32 in fluid communication with a balloon 33 on a distal end of the shaft 31. The balloon occlusion catheter 30 has a guide member 34 comprising a guidewire within lumen 32 and sealingly secured to the distal end of the catheter. In an alternative embodiment (not shown), the guide member 34 is a flexible tip member such as a coil (not shown) secured to the distal end of the shaft 31, as is conventionally known. FIG. 5 illustrates an alternative embodiment in which the embolic protection device 30 in the catheter second lumen 14 is a filter device comprising a guidewire 41 having a trap 42 on a distal end thereof. In the embodiment of FIG. 5, the trap 42 is a collapsible mesh basket, although a variety of suitable filter type embolic protection devices can be used as are conventionally known. In the embodiment of FIG. 5, the device 30 further comprises an elongated shaft 31 with the guidewire 41 disposed in the shaft lumen, and the guidewire 41 can be moved relative to the shaft 31 to reversibly open or collapse the mesh basket.

In the embodiment of FIG. 1, a distal tip member 26 is secured to the distal end of the first polymeric tubular member 22, and is formed of a soft polymeric material to provide an atraumatic distal leading end of the catheter 10. Radiopaque material may be included in or on the distal tip member, or a radiopaque marker band (not shown) provided on the distal tip member, for visualization under x-ray during the medical procedure. The distal tip member 26 defines the distal end of the shaft 11 and the distal port 13 of the first lumen 12. The distal tip member 26 is fusion or adhesively bonded to the distal end of the tubular member 22, and a variety of suitable junctions may be used including a butt joint as shown, or a lap joint.

The distal end of the catheter shaft 11 has a shape configured to facilitate positioning the distal end adjacent to embolic debris trapped by the embolic protection device 30 for removing the debris by suctioning the debris through the first lumen 12. In the embodiment of FIG. 1, the distal end has a truncated shape configured to fit in the space around the sides of the inflated balloon 33 of device 30 between the balloon 33 and the wall of the body lumen 29 to remove embolic debris 50 therefrom. However, the shaft distal end defining the distal port 13 of the first lumen 12 can have a variety of suitable shapes depending on the location of the debris to be removed. For example, FIG. 5 illustrates an alternative embodiment of catheter 10 having a distal end with a tapered shape configured for removing the debris from within the trap 42 of device 30, and FIG. 6 illustrates a distal end with a squared shape. Although not illustrated, the distal end of the shaft 11 can be advanced into the trap 42, with the tapered distal end defining the distal port 13 adjacent to embolic debris in the trap 42 for removal of the debris from the trap 42. The tapered shape facilitates positioning the port 13 at the back (i.e., downstream end) of the trap 42.

When the catheter 10 of the invention is used in an aspiration procedure, the embolic protection device 30 is in place in the body lumen 29, adjacent to an intravascular catheter such as an angioplasty or atherectomy catheter (not shown) during treatment of a stenosed region of the body lumen 29. Dislodged pieces of biological debris which are trapped by the embolic protection device are removed from the body lumen by the catheter 10 of the invention. Specifically, the catheter 10 of the invention is advanced over previously introduced embolic protection device 30 by placing the proximal end of the device 30, extending outside the patient, in the distal port 16 of the second lumen 14 of the catheter 10, and slidably advancing the catheter 10 over the device 30 to position the distal port 13 of the aspiration lumen 12 adjacent to the embolic debris trapped by the device 30.

To the extent not previously discussed herein, the various catheter components may be formed and joined by conventional materials and methods. The shaft can be formed by conventional techniques, such as by extruding and necking materials found useful in intravascular catheters such a polyethylenes, polyvinyl chloride, polyesters, polyamides, polyimides, polyurethanes, and composite materials. Distal tip member 26 is preferably formed of a polymeric material having a lower Shore durometer hardness than the polymeric material forming the first tubular member 22.

The length of the catheter 10 is generally about 30 to about 160 centimeters (cm), and typically about 90 cm for use in the coronary anatomy. The shaft proximal section has an outer diameter (OD) of about 0.030 to about 0.120 inch (0.76 to 3.05 mm), and the shaft distal section has an OD of about 0.015 to about 0.11 inch (0.38 to 2.8 mm). The mandrel typically has a length of about 20 to about 160 cm, and an OD of about 0.2 to about 1.14 mm.

While the present invention has been described herein in terms of certain preferred embodiments, those skilled in the art will recognize that modifications and improvements may be made without departing from the scope of the invention. Moreover, while individual features of one embodiment of the invention may be discussed or shown in the drawings of the one embodiment and not in other embodiments, it should be apparent that individual features of one embodiment may be combined with one or more features of another embodiment or features from a plurality of embodiments. For example, while the catheter distal end having a truncated shape is illustrated with the occlusion balloon embolic protection device, it should be understood that any of the distal end shapes may be used with a variety of embolic protection devices including the embolic protection devices described herein.

What is claimed is:

1. A catheter, comprising an elongated shaft having a proximal end, a distal end, a first lumen extending from a proximal shaft section to the distal end of the shaft with a distal port at the shaft distal end, and a second lumen extending in at least a distal shaft section from a proximal port located distal to the proximal end of the shaft to a distal port located proximal to the distal port of the first lumen.

2. The catheter of claim 1 wherein the shaft distal end has a shape selected from the group consisting of truncated, tapered, and squared.

3. The catheter of claim 1 including a support mandrel longitudinally extending at least in a proximal shaft section.

4. The catheter of claim 3 wherein the support mandrel is in a mandrel lumen radially adjacent to the first lumen.

5. The catheter of claim 3 wherein the support mandrel extends from the proximal end of the shaft to a location spaced proximally from the second lumen distal port.

6. The catheter of claim 1 wherein a proximal shaft section located proximal to the second lumen is formed of a polymeric tubular member having an outer surface defining an outer surface of the shaft and an inner surface defining the first lumen.

7. The catheter of claim 6 wherein the distal end of the shaft is formed of a distal tip member secured to the distal end of the polymeric tubular member.

8. The catheter of claim 7 wherein the distal tip member is formed of a polymeric material having a lower Shore durometer hardness than a polymeric material forming the polymeric tubular member.

9. The catheter of claim 1 wherein the first lumen has an inner diameter larger than an inner diameter of the second lumen.

10. The catheter of claim 1 wherein the second lumen distal port is spaced about 1 to about 30 mm proximally from the first lumen distal port.

11. The catheter of claim 1 wherein the catheter is an aspiration catheter having an adapter on the proximal end configured for connecting a vacuum source in fluid communication with the first lumen.

12. The catheter of claim 1 wherein the catheter is a fluid delivery catheter having an adapter on the proximal end configured for connecting a fluid source in fluid communication with the first lumen.

13. The catheter of claim 1 wherein the second lumen is configured to slidingly receive a device selected from the group consisting of a guidewire and an embolic protection device.

14. The catheter of claim 13 wherein the shaft distal end defining the first lumen distal port has a shape configured to facilitate positioning the distal end directly adjacent to the embolic protection device, and is selected from the group consisting of truncated, tapered, and squared.

15. An aspiration catheter system, comprising:
a) an elongated shaft having a proximal end, a distal end, an aspiration lumen extending from a proximal shaft section to the distal end of the shaft with a distal port at the shaft distal end, and a device lumen extending in at least a distal shaft section from a proximal port to a distal port proximal to the first lumen distal port; and
b) an embolic protection device slidably disposed in the device lumen.

16. The aspiration catheter system of claim 15 wherein the embolic protection device is selected from the group consisting of a guidewire having a trap on a distal section thereof, and an occlusion balloon catheter.

17. The aspiration catheter system of claim 16 wherein the embolic protection device is an occlusion balloon catheter and the shaft distal end defining the first lumen distal port has a truncated shape.

18. The aspiration catheter system of claim 15 wherein the second lumen proximal port is located distal to the proximal end of the shaft.

19. An aspiration or fluid delivery catheter, comprising an elongated shaft having a proximal end, a distal end, an aspiration or fluid delivery lumen extending from a proximal shaft section to the distal end of the shaft with a distal port at the shaft distal end, and a device lumen extending at least in a distal shaft section from a proximal port to a distal port which opens to outside the catheter and which is located proximal to the distal port of the aspiration or fluid delivery lumen.

20. The catheter of claim 19 wherein the proximal port of the device lumen is located at the proximal end of the shaft.

21. The catheter of claim 19 wherein the proximal port of the device lumen is located distal to the proximal end of the shaft.
